(19) United States
(12) Patent Application Publication
(10) Pub. No.: US 2014/0295554 A1
(43) Pub. Date: Oct. 2, 2014

(54) METHOD FOR MANUFACTURING UMBILICAL CORD EXTRACT AND USAGE OF THE SAME

(71) Applicants: Sun Mi Kim, Gyeonggi-do (KR); Youngjun Lee, Seoul (KR); Yong Soo Choi, Gyeonggi-do (KR)

(72) Inventors: Sun Mi Kim, Gyeonggi-do (KR); Youngjun Lee, Seoul (KR); Yong Soo Choi, Gyeonggi-do (KR)

(73) Assignee: CHA BIO & DIOSTECH CO., LTD., Seoul (KR)

(21) Appl. No.: 14/232,448

(22) PCT Filed: Jul. 11, 2012
(86) PCT No.: PCT/KR2012/005514

§ 371 Date: Jan. 13, 2014

(51) Int. Cl. C12N 5/074

(52) U.S. Cl. CPC C12N 5/0607
USPC 435/408

(57) ABSTRACT

The present invention provides a method for effectively extracting useful ingredients from an umbilical cord. The present invention provides an umbilical cord extract including the useful ingredients. The umbilical cord extract, according to the present invention, can be used as a serum substitute for cultivating ordinary cells and stem cells from an animal. Also, the umbilical cord extract, according to the present invention, can be used for a filler and a dressing for tissue restoration, and for a cosmetic composition for improving the skin. In addition, the present invention relates to a composition for a medium for separating and stem cells derived from tissue, such as an umbilical cord and fatty tissue, and to a method for separating and cultivating stem cells derived from the tissue using the same.

### RELATED APPLICATIONS

This application claims the benefit of Korean Patent Application No. 10-2011-0068761, filed on Jul. 12, 2011, and Korean Patent Application No. 10-2011-0068261, filed on Jul. 11, 2011, in the Korean Intellectual Property Office, the disclosures of which are incorporated herein in their entirety by reference.

### BACKGROUND

1. Field

One or more embodiments of the present invention relate to a method of preparing an umbilical cord extract, a serum substitute of the umbilical cord extract, a composition for separating and culturing stem cells, and uses of a filler for wound healing.

2. Description of the Related Art

Since the establishment of an extracorporeal animal cell culture system, serum has been conventionally used to facilitate the proliferation of animal cells. The serum, which has been widely used for the survival and proliferation of primary cells and established cell lines, is a mixture which only has a portion of the composition thereof known, and thus, biological activity of the serum in a cell culture is not completely known. Also, fetal bovine serum (FBS) is collected from fetal bovine and thus, FBS has hazardous factors such as mycoplasma, viruses, prions, bacterial mitogens, hormones, extraneous proteins, growth factors, and proteases. Furthermore, the quality of a composition of FBS may vary depending on equipment, technical standards, and production lots of a supplier. Basal media reported thus far are known to enable the growth of specific cells; cell growth factors, adhesion factors, or the like used instead of the serum are expensive; and the growth and metabolite production in a basal medium are known to be less stable than those in a medium including the serum (Cruz J. H. et al., Cytotechnology, 26: 59-64(1998) and Hee-Chan Lee, A Basal medium in an Animal Cell Culture, Biotechnology News, 2(3):242-252(1994)).

Also, a medium, including FBS, is conventionally used to continuously culture adult stem cells in an undifferentiated state and an animal-derived protein source, including FBS, is used during the culturing, which may cause stability problems such as contamination between species during the development of a stem cell treatment for clinical applications. Accordingly, the clinical applications of the stem cells obtained from the conventional culturing method have many limitations.

As a method of circumventing the problems of the use of the animal-derived protein source (i.e., FBS), a method of using a basal medium and a method of using a medium including human serum may be used. The method of using the basal medium includes culturing stem cells in a medium containing a large amount of cytokines such as growth hormones and thus, the method is cumbersome and uneconomical. Also, the method of using the medium including the human serum also includes the use of a large amount of cytokines prepared by a recombination method and the use of expensive human serum as a protein source for culturing and thus, the method is economically inefficient.

Meanwhile, stem cells may be categorized into adult stem cells that are found in various tissues and organs in adults and embryonic stem cells that may be obtained from cells in a blastodermic stage. The embryonic stem cells have pluripotency for differentiating into various cells such as nerve cells, blood cells, and pancreatic cells; however, the embryonic stem cells are obtained from a human embryo, and thus, are subject to ethical problems. Accordingly, adult stem cells, which are free of ethical problems, may be easily separated and cultured, and are capable of differentiating into various cells, and thus, the adult stem cells are receiving much attention as a material for a cell therapy product.

The adult stem cells may be separated from various tissues and are undifferentiated stem cells that may differentiate into various tissue cells such as fat cells, bone cells, cartilage cells, heart cells, liver cells, and neural cells. Among these, bone marrow-derived mesenchymal stem cells (BM-MSCs) are a representative example of the adult stem cells.

However, as the age of a stem cell donor increases, the number and proliferation potency of the BM-MSCs decrease and bone marrow extraction causes much pain to the donor. Accordingly, attempts are being made to separate mesenchymal cells from different tissues. Due to various recent reports reporting that the mesenchymal cells may be separated from peripheral blood, an umbilical cord, placenta, and umbilical cord blood of an adult or embryo, the mesenchymal cells obtained from various tissues are receiving much attention as a source of a new cell therapy product.

An umbilical cord includes blood vessels and connective tissues known as Wharton's jelly surrounding the blood vessels. During pregnancy, a length of the umbilical cord may be about 30 cm to 60 cm, and the weight of the umbilical cord may be about 40 g to about 50 g. Also, the umbilical cord includes a sufficient amount of nutrients for supplying to a fetus, stem cells and precursor cells. Recently, it has been reported that cells derived from the umbilical cord have properties of BM-MSCs. Similar to the bone marrow and other tissue-derived mesenchymal stem cells, the umbilical cord-derived stem cells express cell surface proteins such as CD73, CD90, CD105, CD10, CD13, CD29, CD44, and HLA-ABC, but do not express cell surface proteins such as CD34 and CD45, which are hematopoietic stem cell markers, and CD14, CD31, CD33, and HLA-DRα, which are histocompatibility antigens. Furthermore, the stem cells separated from the umbilical cord have been reported to simultaneously express Oct4, Sox2, Nanog, and the like, which are embryonic stem cell markers.

The umbilical cord-derived stem cells may differentiate into bone cells, cartilage cells, and fat cells and have better mitotic activity than bone marrow or fat-derived stem cells during an in vitro culturing. It has also been reported that the umbilical cord-derived stem cells may differentiate into cardiac myocytes and nerve cells. In this regard, the umbilical cord is a tissue that may supply stem cells for clinical applications and may be used as a cell therapy product.

However, a great number of the umbilical cord-derived stem cells are needed to use the umbilical cord-derived stem cells efficiently. However, there is a limit to the number of stem cells that may be obtained from the umbilical cord and many of the stem cells lose differentiation potency during culturing. However, there is no known method of efficiently separating and culturing the umbilical cord-derived stem cells in a basal medium. While researching about solutions to resolve this problem, it has been identified that the umbilical cord-derived stem cells may be efficiently separated and cultured when an umbilical cord-derived extract is used and thus, the present invention was completed.

### SUMMARY

One or more embodiments of the present invention include a method of preparing an umbilical cord extract.

One or more embodiments of the present invention include a serum substitute including the umbilical cord extract.

One or more embodiments of the present invention include a cell culture medium including the serum substitute.

One or more embodiments of the present invention include a method of separating umbilical cord-derived stem cells including the umbilical cord extract from an umbilical cord and a method of culturing the umbilical cord-derived stem cells.

Additional aspects will be set forth in part in the description which follows and, in part, will be apparent from the description, or may be learned by practice of the presented embodiments.

### BRIEF DESCRIPTION OF THE DRAWINGS

These and/or other aspects will become apparent and more readily appreciated from the following description of the embodiments, taken in conjunction with the accompanying drawings of which:

FIG. 1 is a graph showing total amount of protein eluted according to stirring time;

FIG. 2 is a graph showing a comparison between total amounts of proteins eluted when a medium is changed and when the medium is not changed, according to time;

FIGS. 3 and 4 are showing total amounts of proteins eluted according to the size of an umbilical cord;

FIGS. 5 and 6 are images showing total amounts of proteins eluted according to pH of buffers and SDS-PAGE results for identifying differences between eluted proteins;

FIGS. 7 and 8 are images showing total amounts of proteins eluted according to elution methods;

FIGS. 9 and 10 are images showing differences between proteins eluted from a fresh umbilical cord tissue and from a frozen umbilical cord tissue;

FIG. 11 is an image showing total amounts of proteins eluted according to the amount of buffer;

FIGS. 12, 13, and 14 are graphs showing quantitative analysis results of umbilical cord extract (UCE) cytokines;

FIGS. 15, 16, 17, and 18 are graphs and images showing cell proliferation according to a culturing additive in a basal medium;

FIGS. 19 and 20 are graphs showing results of culturing bone marrow and umbilical cord-derived stem cells in a basal medium including an umbilical cord extract, treating mesenchymal stem cell markers, and performing a fluorescence activated cell sorter (FACS) analysis;

In this regard, x-axis indicates intensity and y-axis indicates the number of cells (count). CD markers written on the graphs may be distinguished based on changes in the x-axis and the y-axis. The drawings show images comparing the cells grown in a medium including FBS to the cells grown in a medium including the umbilical cord extract;

FIGS. 21 and 22 are images showing an increased inflow of surrounding tissue-derived cells into a filler when an umbilical cord extract (UCE) is included therein, due to a subcutaneous injection of a hyaluronic acid derivative (HAD) filler including the UCE into a mouse and then dying with Hematoxylin & Eosin;

FIG. 23 shows proliferation of umbilical cord-derived stem cells according to a concentration of an umbilical cord extract in a basal medium according to an embodiment;

FIG. 24 shows increased adhesion and proliferation (culturing) of umbilical cord-derived stem cells in a culture dish coated with umbilical cord-derived collagen according to an embodiment;

FIG. 25 shows results of comparing the numbers of umbilical cord-derived stem cells cultured in culture dishes coated with collagen according to concentrations of the collagen and then comparing the number of umbilical cord-derived stem cells proliferated after 2 days;

FIG. 26 shows images comparing a conventional method of separating umbilical cord-derived stem cells to a method of separating umbilical cord-derived stem cells according to an embodiment;

FIG. 27 shows images of cells that are separated and cultured according to 6 methods shown in FIG. 26;

FIG. 28 shows the total number of cells recovered 15 days after separating umbilical cord-derived stem cells;

FIG. 29 shows comparative analysis results of immune indicators separated and cultured according to the six separation and culturing methods shown in FIG. 26;

In this regard, x-axis indicates intensity and y-axis indicates the number of cells (count). CD markers written on the graphs may be distinguished based on changes in the x-axis and the y-axis;

FIG. 30 shows results of cytokine arrays performed with respect to 507 different types of human cytokines, chemokines, growth factors or the like included in an extract separated from umbilical cords from three different donors and 10 cytokines that are included in greatest amounts for each umbilical cord;

FIG. 31 is a comparative quantitative graph showing a material that is commonly included in an extract separated from three different umbilical cords. The cytokines that are commonly included in greatest amounts are IGFBP-7 and lipocalin-1;

FIG. 32 sequentially shows 62 types of human cytokines identified in three different types of umbilical cords shown in the order of the cytokines that are included the greatest amount to the smallest amount;

FIG. 33 describes functions of 10 cytokines having functions that are well known in a human body;

FIGS. 34 and 35 show results of experiments comparing stemness maintenance between stem cells that are continuously subcultured in an umbilical cord extract and in a medium including 10% FBS, wherein doubling time (Td) values of the stem cells during an initial subculture and after 10 cycles of subcultures are compared;

FIG. 36 shows that umbilical cord derived stem cells (UC-MSCs) separated from an umbilical cord tissue by using an umbilical cord extract express embryonic stem cell (ESC) specific markers;

FIG. 37 shows that all of stem cells separated by using an umbilical cord extract of a tissue obtained from three different donors are positive for CD29, CD73, CD90, CD105, and CD166, which are mesenchymal stem cell specific cell surface markers and are negative for CD34 and CD45;

FIG. 38 shows that during culturing of stem cells by using an umbilical cord extract, the embryonic stem cell specific markers that have been identified during an initial stage of stem cell separation are no longer expressed when FBS is added to a culture medium but are maintained when the umbilical cord extract is added to the culture medium.

### DETAILED DESCRIPTION

Reference will now be made in detail to embodiments, examples of which are illustrated in the accompanying drawings, wherein like reference numerals refer to like elements throughout. In this regard, the present embodiments may have different forms and should not be construed as being limited to the descriptions set forth herein. Accordingly, the embodiments are merely described below, by referring to the figures, to explain aspects of the present description. As used herein, the term “and/or” includes any and all combinations of one or more of the associated listed items. Expressions such as “at least one of,” when preceding a list of elements, modify the entire list of elements and do not modify the individual elements of the list.

Provided is a method of preparing an umbilical cord extract (UCE).

Provided is a method of preparing a mammalian UCE, according to an embodiment, the method including cutting an umbilical cord; putting the umbilical cord into a buffer; stirring the umbilical cord impregnated in the buffer; and centrifuging a product obtained from the stirring to obtain a supernatant as the UCE.

Provided is a method of ameliorating problems of a conventional method to separate growth factors, cytokines, chemokines, and glycoproteins such as glycaosminoglycans (GAGs) bound to an extracellular matrix by a relatively simple process to obtain useful ingredients for a cell in an active and a naturally-occurring state as much as possible.

The method of the present invention may be described in detail according to the following processes:

First, the method includes cutting an umbilical cord.

According to an embodiment, first, the umbilical cord is cut. According to an embodiment, cutting the umbilical cord increases a contact surface between a buffer and an umbilical cord tissue to facilitate the elution of useful materials from the umbilical cord tissue.

The umbilical cord that may be used in the embodiment includes umbilical cords of various mammals. The mammal may preferably be a human, pig, horse, cow, mouse, rat, hamster, rabbit, goat, and sheep, and more preferably a human, pig, horse, and cow, and most preferably, a human.

The cutting of the umbilical cord may be performed by various methods known in the art.

According to an embodiment, the umbilical cord is cut into a length of about 0.5 cm to about 3.0 cm, and more preferably about 0.7 cm to about 2.5 cm, and more preferably about 1 cm to about 2 cm.

Particularly, the UCE may preferably be extracted from Wharton's jelly in the umbilical cord tissue. In this regard, the method may additionally include removing blood and/or blood vessels from the umbilical cord.

Thereafter, the method may include putting the cut umbilical cord into a buffer.

The cut umbilical cord is put into a buffer. The buffer used in the art may be any buffer having buffering power and may be, for example, sodium acetate, sodium phosphate, glycin-HCl, Tris-HCl, and phosphate buffered saline (PBS). More particularly, PBS may be used at a pH of about 2 to about 11, more preferably, at about 4 to about 10, more preferably, at about 5 to about 8, and more preferably, at about 6.8 to about 7.6.

An amount of the buffer for immersing the cut umbilical cord is not particularly limited and may preferably be a buffer having a weight that is about 2 to about 5 times as great, more preferably, about 2 to about 4 times as great, and more preferably about 2.5 times to about 3.2 times as great as the weight of the umbilical cord. Also, the cut umbilical cord may be washed with a suitable solution (for example, a buffer) before putting the cut umbilical cord into the buffer.

Thereafter, the method may include stirring the umbilical cord impregnated with the buffer.

According to an embodiment, the stirring may be performed at a temperature of about 4° C. to about 10° C. and preferably at about 4° C. to about 6° C. Also, the stirring may be performed for about 7 hours to about 24 hours, preferably for about 12 hours to about 24 hours, and more preferably for about 18 hours to about 24 hours.

The stirring may be performed by using various methods known in the art, and a magnetic bar may be used for the stirring.

Thereafter, a product obtained from stirring is centrifuged to obtain a supernatant as the UCE.

A product obtained from the stirring is centrifuged to finally obtain a supernatant as the UCE. The supernatant includes various useful proteins such as growth factors and cytokines bound to the extracellular matrix.

According to an embodiment, the centrifugation may be performed at about 3,000 rpm to about 6,000 rpm and more preferably at about 4,000 rpm to about 4,500 rpm. The centrifugation may be performed at a temperature of about 4° C. to about 10° C. and more preferably at about 4° C. to about 6° C. According to an embodiment, the centrifugation may be performed for about 2 minutes to about 30 minutes, preferably for about 5 minutes to about 20 minutes, and more preferably for about 10 minutes to about 15 minutes.

Provided is the UCE prepared according to the method according to an embodiment.

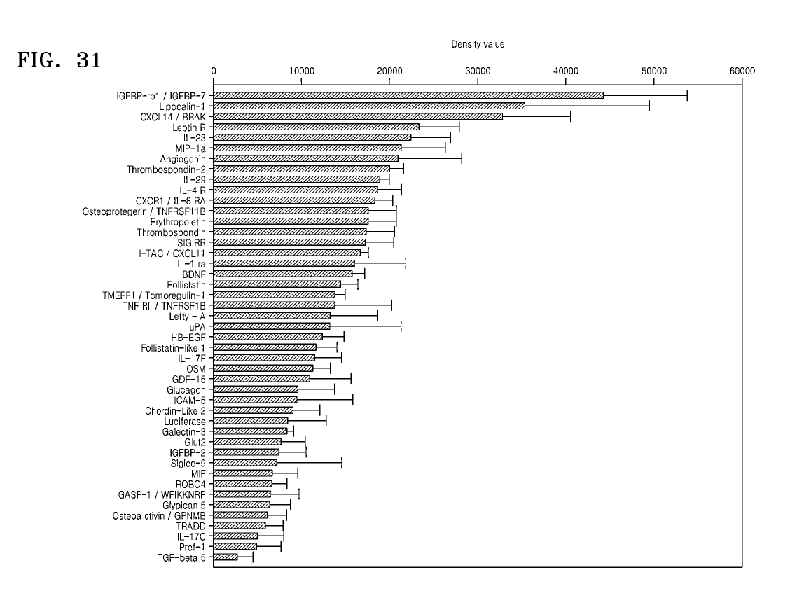

According to an embodiment, the UCE includes insulin-like growth factor binding protein-7 (IGFBP-7), Lipocallin-1, CXCL14, Leptin R, IL-23, MIP-1a, Angiogenin, Thrombospondin-2, IL-29, IL-4R, and the like (FIG. 31) as primary ingredients.

Primary cytokines of the UCE are well known for effects related to anti-angiogenesis, anti-apoptosis, growth, and inflammation.

Provided is a serum substitute including the UCE.

The term “serum” as used herein refers to remaining portions of plasma after removing cellulose. The serum is conventionally used to facilitate the proliferation of animal cells since the establishment of an in vitro animal cell culturing system.

The term “serum substitute” as used herein refers to a material that may be used to obtain the same or similar effects as serum and may be a material that may obtain the same or excellent effects without using serum such as fetal bovine serum (FBS).

The UCE may be UCE obtained according to a conventional method; however, the UCE may preferably be UCE obtained according to embodiments described above. More particularly, it is preferable to remove blood from the umbilical cord to exclude serum ingredients.

The serum substitute may be applied to all fields in which serum may be applied. More particularly, the serum substitute may be used in culturing cells, and more preferably be used in culturing animal cells. The stem cells may be any type of stem cells such as adult stem cells, mesenchymal stem cells, dedifferentiated stem cells, or tissue-derived stem cells.

Provided is a cell culture medium including the serum substitute.

The cell culture medium according to an embodiment may include various animal cells, preferably mammalian cells, and more preferably human, pig, horse, cow, mouse, rat, hamster, rabbit, goat, and sheep cells, more preferably human, pig, horse and cow cells, and most preferably human cells.

The cell culture medium according to an embodiment may be applied to a stem cell culture. The stem cells as used herein are not limited and are cells having stem cell properties, i.e., cells capable of differentiation, unlimited proliferation, and differentiation into specific cells. The stem cells may include pluripotent stem cells and multipotent stem cells including embryonic stem (ES) cells and embryonic germ (EG) cells. Preferably, the stem cells may be umbilical cord-derived stem cells (UC-MSCs).

The cell culture medium according to an embodiment is a serum substitute and may include basic ingredients of a medium for culturing animal cells in addition to the UCE. For example, the cell culture medium of the present invention may be prepared based on Eagles's minimum essential medium (EMEM) (EMEM, Eagle, H. Science 130:432(1959)), α-MEM (Stanner, C. P. et al., Nat. New Biol. 230:52(1971)), Iscove's MEM (Iscove, N. et al., J. Exp. Med. 147:923(1978)), medium 199 (Morgan et al., Proc. Soc. Exp. Bio. Med., 73:1(1950)), CMRL 1066, RPMI 1640 (Moore et al., J. Amer. Med. Assoc. 199:519(1967)), F12 (Ham, Proc. Natl. Acad. Sci. USA 53:288(1965)), F10 (Ham, R. G. Exp. Cell Res. 29:515(1963)), Dulbecco's modification of Eagle's medium (DMEM) (DMEM, Dulbecco, R. et al., Virology 8:396(1959)), a mixture of DMEM and F12 (Barnes, D. et al., Anal. Biochem. 102:255(1980)), Way-mouth's MB752/1 (Waymouth, C. J. Natl. Cancer Inst. 22:1003(1959)), McCoy's 5A (McCoy, T. A., et al., Proc. Soc. Exp. Biol. Med. 100:115(1959)), and MCDB series (Ham, R. G. et al., In Vitro 14:11(1978)).

According to another embodiment, provided is a filler for tissue restoration, the filler including the UCE.

The wording “filler for tissue restoration” refers to a medical composition or a cosmetic composition used for effectively concealing wrinkles and fine lines of skin.

The filler for tissue restoration, according to an embodiment, includes basic ingredients of a filler, preferably collagen, hyaluronic acid, polyacrylamide gel, artecoll, autologen (autologous collagen), or polymethacrylate, and a collagen mixture in addition to the UCE.

The filler according to an embodiment may include wax, elastomer, higher alcohol, surfactant, oil, powder, humectant, waterborne polymer, skin protectant, antiseptic and/or scent.

Also, the UCE may be obtained by a conventional method and may preferably be obtained through the embodiments described above. Particularly, it is preferable to remove blood from the umbilical cord to exclude serum ingredients.

According to another embodiment, provided is a dressing including the UCE.

The term “dressing” as used herein refers to a pharmaceutical composition applied to a part of a human or animal body for a clinical or aesthetic skin treatment. Preferably, the dressing is for treating damaged skin, skin lesions, and random interruptions on a skin surface (for example, skin ulcers, burns, cuts, punctures, ripped wounds, blunt injuries, acne lesions, and furuncles). The dressing may include a patch, a plaster, a bandage, or gauze for thoroughly transporting medicine. The dressing may preferably be applied to internal tissue and external tissue of the body, and more preferably to the surface of the body.

Also, the UCE may be obtained by a conventional method and may preferably be obtained through the embodiments described above. More particularly, it is preferable to remove blood from the umbilical cord to exclude serum ingredients.

According to another embodiment, provided is an anti-wrinkle or anti-aging cosmetic composition including the UCE.

The cosmetic composition according to an embodiment may be used for improving various skin conditions. Preferably, the cosmetic composition of the present invention may be effective for anti-wrinkling and anti-aging.

The ingredients included in the cosmetic composition according to an embodiment are active ingredients that are conventionally included in a cosmetic composition in addition to growth factors, and the ingredients may be a conventional supplement such as a stabilizer, a dissolution agent, a vitamin, a dye, and a perfume, and a carrier.

The cosmetic composition according to an embodiment may be prepared as any conventional formulation in the art, which may be, but is not limited to a solution, suspension, emulsion, paste, gel, cream, lotion, powder, soap, surfactant-containing cleanser, oil, powder foundation, emulsion foundation, wax foundation, and spray and more particularly, skin toner, nourishing skin toner, nourishing cream, massage cream, essence, eye cream, cleansing cream, cleansing foam, cleansing water, facial mask, spray, or powder.

When the formulation according to an embodiment is a paste, cream, or gel, animal oil, vegetable oil, wax, paraffin, starch, tragacanth, cellulose derivative, polyethylene glycol, silicone, bentonite, silica, talc, or zinc oxide may be used as a component of the carrier.

When the formulation according to an embodiment is powder or spray, lactose, talc, silica, aluminum hydroxide, calcium silicate, or polyamide powder may be used as a component of the carrier. More particularly, when the formulation is a spray, a propellant such as chloro-fluoro hydrocarbon, propane/butane, or dimethyl ether may be included as the component of the carrier.

When the formulation according to an embodiment is a solution or an emulsion, a solvent, a dissolution agent, or a demulsifier may be used as the component of the carrier, and examples thereof include water, ethanol, isopropanol, ethyl carbonate, ethyl acetate, benzyl alcohol, benzyl benzoate, propylene glycol, 1,3-butyl glycol oil, glycerol aliphatic ester, polyethylene glycol, or fatty acid esters of sorbitan.

When the formulation according to an embodiment is a suspension, a liquid diluent such as water, ethanol, and propylene glycol; a suspending agent such as ethoxylated isostearyl alcohol, polyoxyethylene sorbitol ester, and polyoxyethylene sorbitan ester; microcrystalline cellulose; aluminum meta-hydroxide; bentonite; agar; or tragacanth may be used.

When the formulation according to an embodiment is a surfactant-containing cleanser, aliphatic alcohol sulfate, aliphatic alcohol ether sulfate, sulfosuccinate monoester, isethionate, imidazolinium derivative, methyl taurate, sarcosinate, fatty acid amide ether sulfate, alkyl amido betaine, aliphatic alcohol, fatty acid glyceride, fatty acid diethanolamide, vegetable oil, ethoxylated glycerol fatty acid ester, lanolin derivative, or ethoxylated glycerol fatty acid ester may be used as the component of the carrier.

According to another embodiment, provided is a method of separating stem cells from an umbilical cord.

According to another embodiment, provided is a method of separating stem cells from a mammalian umbilical cord, the method including putting an umbilical cord tissue morcellated with a cell culture medium composition including a mammalian UCE into a cell culture container; treating the umbilical cord tissue with a stem cell separation enzyme; and separating stem cells from the umbilical cord tissue.

The mammal may be a human, pig, horse, cow, mouse, rat, hamster, rabbit, goat, or sheep.

The tissue may be selected from the group consisting of fat, an umbilical cord, a liver, and periosteum.

The cell culture container may be a cell culture container coated with cell adhesion proteins. In this regard, the cell adhesion protein may be, but is not limited to a mammalian umbilical cord-derived collagen, gelatin, fibronectin, laminin, or poly-D-lysin.

The mammalian umbilical cord-derived collagen may be prepared by a method of preparing the mammalian umbilical cord-derived collagen, the method including (i) pulverizing the mammalian umbilical cord tissue treated with hydrogen peroxide; (ii) treating the umbilical cord tissue with acetic acid and pepsin and then centrifuging the same; (iii) setting a pH of a supernatant obtained from the centrifugation and adding NaCl thereto to immerse collagen; and (iv) separating the immersed collagen.

In the method of separating stem cells from the mammalian umbilical cord according to an embodiment, it is preferable that process (ii) be performed about 1 to about 3 days after performing process (i).

The stem cell separation enzyme of process (ii) may be a collagenase, and more preferably, type I collagenase. More preferably, in process (ii), the type I collagenase is included up to an amount of about 180 U/ml to about 220 U/ml and may be treated for about 2 hours to about 6 hours.

Also, it is preferable that the UCE be prepared according to the method described in the embodiment above.

According to another embodiment, provided is a method of culturing stem cells by using an UCE.

According to another embodiment, provided is a method of culturing stem cells, the method including adding UCE to a stem cell culture medium and culturing stem cells by using the stem cell culture medium in a cell culture container.

The cell culture container may be coated with cell adhesion proteins. The stem cell may be a tissue-derived stem cell. Also, the stem cell may be an animal stem cell, and more preferably, a human-derived stem cell. Also, the stem cell may be any type of stem cell such as an adult stem cell, a mesenchymal stem cell, a dedifferentiated stem cell, and a tissue-derived stem cell.

Also, the cell adhesion protein may be collagen, gelatin, fibronectin, laminin, or poly-D-lysin, but the cell adhesion protein is not limited thereto.

The tissue may be selected from the group consisting of fat, an umbilical cord, a liver, and periosteum, but the tissue is not limited thereto.

The stem cell culture medium may not include serum.

Also, the stem cell may be any animal stem cell, may be an ES cell, adult stem cell, and dedifferentiated stem cell, and may be a cell in which at least one gene from the group consisting of Oct4, Sox2, KLF4, and Nanog is expressed. More particularly, the cell may continuously express at least one gene from the group consisting of Oct4, Sox2, KLF4, and Nanog, which are ES cell specific genes, even when the cells are subcultured.

Also, the stem cell may be selectively positive for CD29, CD73, CD90, CD105, and CD166, and may be selectively negative for CD34 and CD45.

### EXAMPLE 1

### Comparing Total Amounts of Proteins Eluted According to Stirring times

An umbilical cord was cut into a length of about 0.5 cm to about 2.0 cm, washed with PBS (pH 7.0) twice or more, PBS was added thereto at a weight that is three times as great as weight of the umbilical cord, stirred at a temperature of 4° C. for about 30 minutes to about 24 hours without replacing PBS, and then stirred for about 24 hours to about 200 hours while replacing the PBS to obtain an intermediate product. A supernatant collected from centrifuging the intermediate product at 4,500 rpm and at 4° C. for 10 minutes was used as an umbilical cord-derived extract (umbilical cord extract, UCE) and then Bradford analysis was performed to quantify protein.

As stirring time increased, the amount of protein eluted increased as well, an average of about 2.5 ug/ml of protein was eluted after 7 hours of stirring and an average of about 2.7 ug/ml of protein was eluted after 24 hours of stirring (FIG. 1).

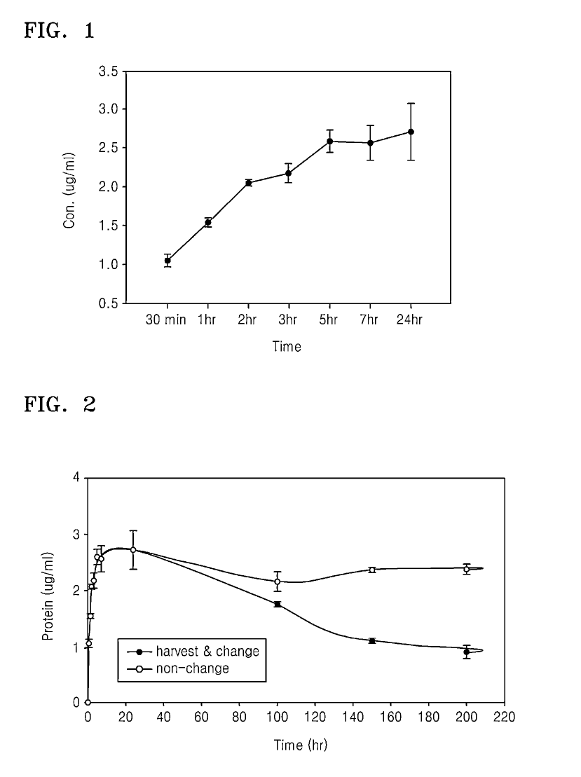

Also, from the starting point of stirring to 24 hours after stirring, the amount of protein eluted increased as time passed; however, when (the UCE) was stirred while replacing the PBS, the amount of protein eluted rapidly decreased after 60 hours from the starting point of stirring (FIG. 2).

### EXAMPLE 2

### Comparing total Amounts of Proteins Eluted According to the sizes of Umbilical Cords

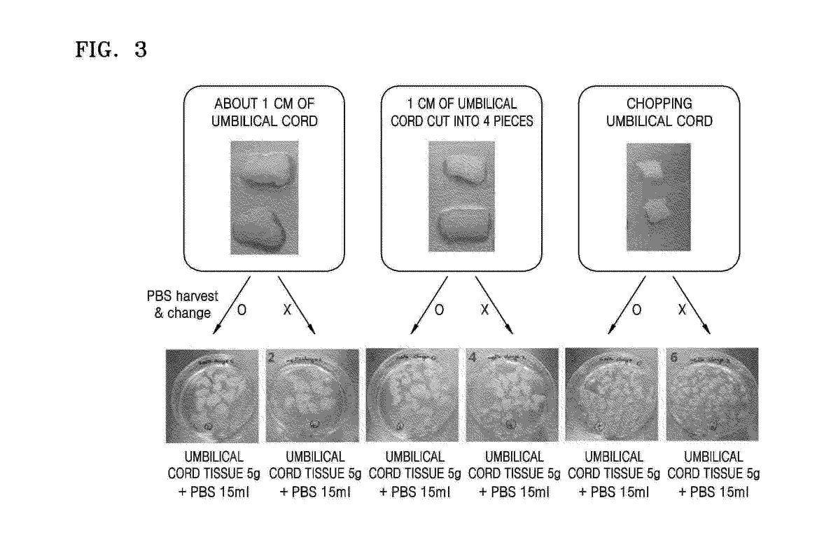

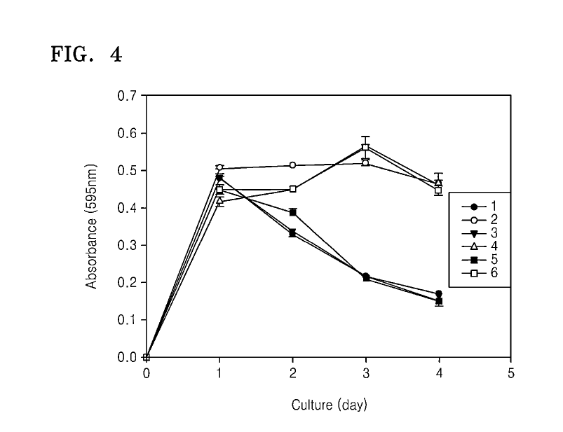

An umbilical cord was cut into a length of about 0.5 cm to about 2.0 cm (FIG. 3), washed with PBS (pH 7.0) twice or more, PBS was added thereto at a weight that is three times as great as the weight of the umbilical cord, and then stirred at a temperature of 4° C. for 4 days. A supernatant collected from centrifuging the intermediate product at 4,500 rpm and at 4° C. for 10 minutes was used as an umbilical cord-derived extract (UCE) and then Bradford analysis was performed to quantify protein. The total amount of protein eluted according to the size of the umbilical cord did not change much and the total amount of protein eluted decreased rapidly when the PBS was replaced while stirring (FIG. 4).

### EXAMPLE 3

### Comparing Total Amounts of Proteins Eluted According to pH of a Buffer

An umbilical cord was cut into a length of about 0.5 cm to about 2.0 cm, PBS (pH 2, pH 7, or pH 11) was added thereto at a weight that is three times as great as the weight of the umbilical cord, and then stirred at a temperature of about 4° C. for 24 hours to obtain an intermediate product. The intermediate product was centrifuged at 4,500 rpm, at a temperature of 4° C. for 10 minutes, and a supernatant obtained therefrom was used as an umbilical cord-derived extract (UCE). Then, Bradford analysis was performed to quantify protein.

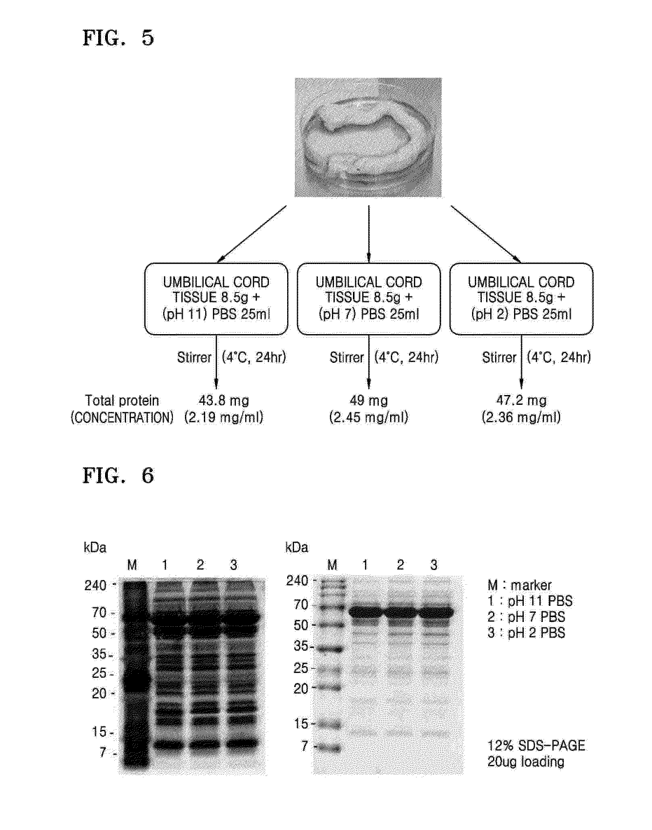

When PBS at pH 2 was used, a total of 47.2 mg (2.36 mg/ml) of protein, and when PBS at pH 7 was used, a total of 49 mg (2.45 mg/ml) of protein, and when PBS at pH 11 was used, a total of 43.8 mg (2.19 mg/ml) of protein were eluted. Thus, it may be concluded that the total amount of protein eluted according to the pH of the PBS does not differ much (FIG. 5), but when the PBS at pH 2 was used, a viscosity of the product after stirring increased.

Also, when sodium dodecyl sulfate-polyacrylamide gel coumassie staining was performed on each UCE that has been protein quantified, the protein bands of the UCEs were similar to each other (FIG. 6).

### EXAMPLE 4

### Comparing Total Amounts of Proteins Eluted According to Elution Methods

An umbilical cord was cut into a length of about 0.5 cm to about 2.0 cm and then washed with PBS (pH 7.0) twice or more, 15 ml of PBS was added to about 8 g of the umbilical cord and then homogenized, stirred (at a temperature of 4° C. for 24 hours), or incubated (at a temperature of 37° C. for 24 hours) to obtain an intermediate product. The intermediate product was centrifuged at 4,500 rpm, at 4° C. for 10 minutes, and the supernatant obtained therefrom was used as an UCE, and protein was quantified by Bradford analysis.

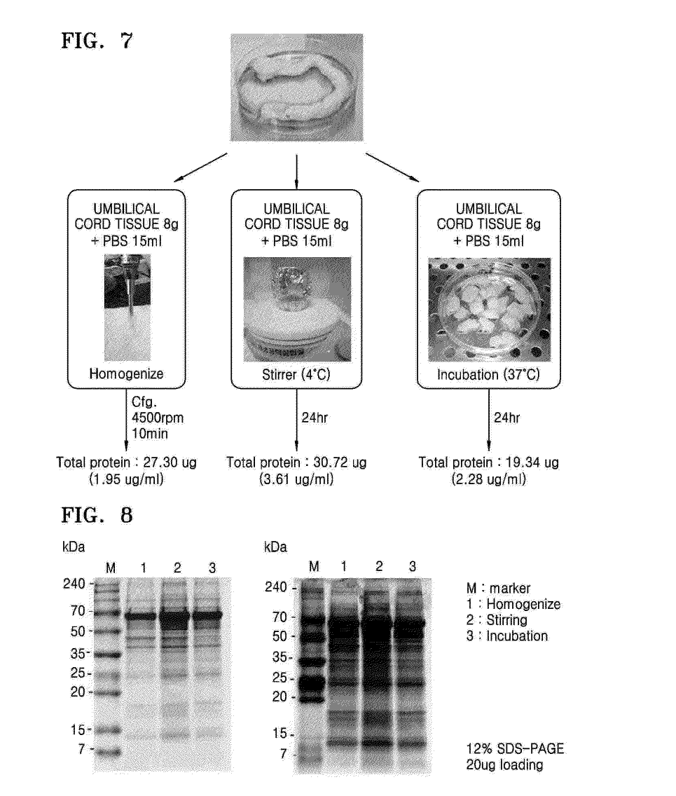

When homogenized, a total of 27.3 mg (1.95 mg/ml) of protein, when stirred, a total of 30.72 mg (3.61 mg/ml) of protein, and when cultured, a total of 19.34 mg (2.28 mg/ml) of protein were eluted. As a result, it may be concluded that the greatest amount of protein was eluted when stirred at a temperature of 4° C. (FIG. 7).

Also, when protein-quantified UCEs under conditions described above were sodium dodecyl sulfate-polyacrylamide gel coumassie stained, the protein bands of the UCEs were similar to each other (FIG. 8).

Accordingly, it was identified that stirring at a temperature of 4° C. is a method of obtaining a great amount of protein while preventing protein denaturation and decreased protein stability, which may occur during the homogenization and culturing at a temperature of 37° C.

### EXAMPLE 5

### Comparing Total Amounts of Proteins Eluted According to Methods of Storing Umbilical Cord Tissues

An umbilical cord was cut into a length of about 0.5 cm to about 2.0 cm and then washed with PBS (pH 7.0) twice or more, and about 33 ml of PBS was added to about 11 g of the umbilical cord, stirred (at a temperature of 4° C. for 24 hours) or frozen (at −80° C. for 6 days), and then stirred (at 4° C. for 24 hours) to obtain an intermediate product. The intermediate product was centrifuged at 4,500 rpm, at 4° C. for 10 minutes, and the supernatant obtained therefrom was used as an UCE, and protein was quantified by Bradford analysis.

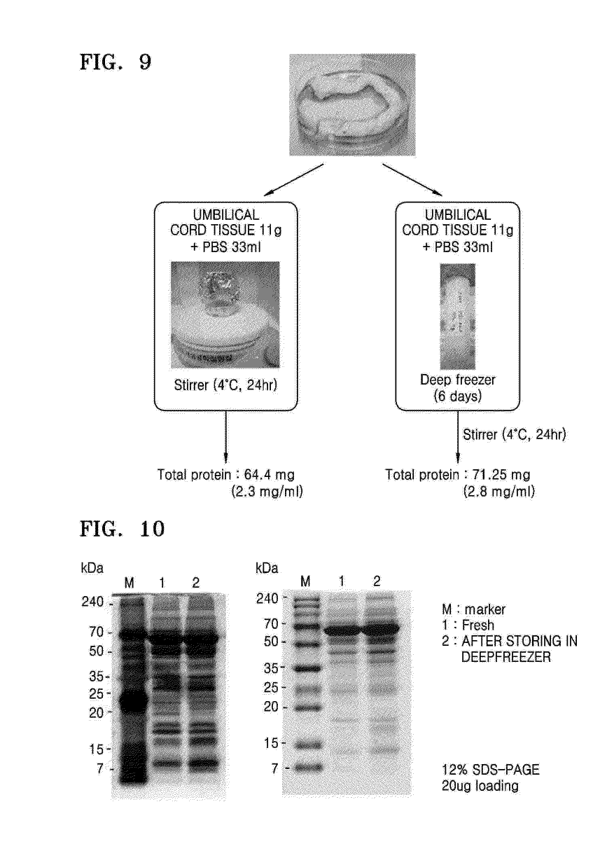

For the frozen umbilical cord, about 7 mg of protein was additionally eluted compared to a fresh umbilical cord (FIG. 9). However, after sodium dodecyl sulfate-polyacrylamide gel coumassie staining of UCEs that have been protein quantified under the conditions described above, protein bands of the UCEs were similar to each other (FIG. 10).

### EXAMPLE 6

### Comparing Total Amounts of Proteins Eluted According to the Amount of Buffer

An umbilical cord was cut into a length of about 0.5 cm to about 2.0 cm and then washed with PBS (pH 7.0) twice or more, about 22 ml (1:2), about 33 ml (1:3), or about 55 ml (1:5) of the PBS was added to about 11 g of the umbilical cord, and then stirred at 4° C. for 24 hours. The umbilical cord was centrifuged at 4,500 rpm, at 4° C. for 10 minutes, and the supernatant obtained therefrom was used as UCE, and protein was quantified by Bradford analysis.

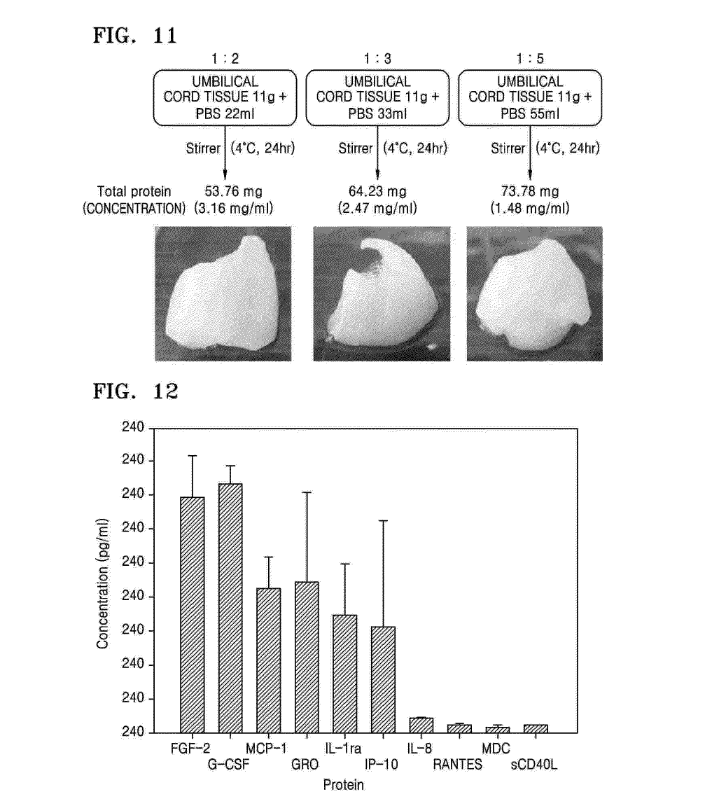

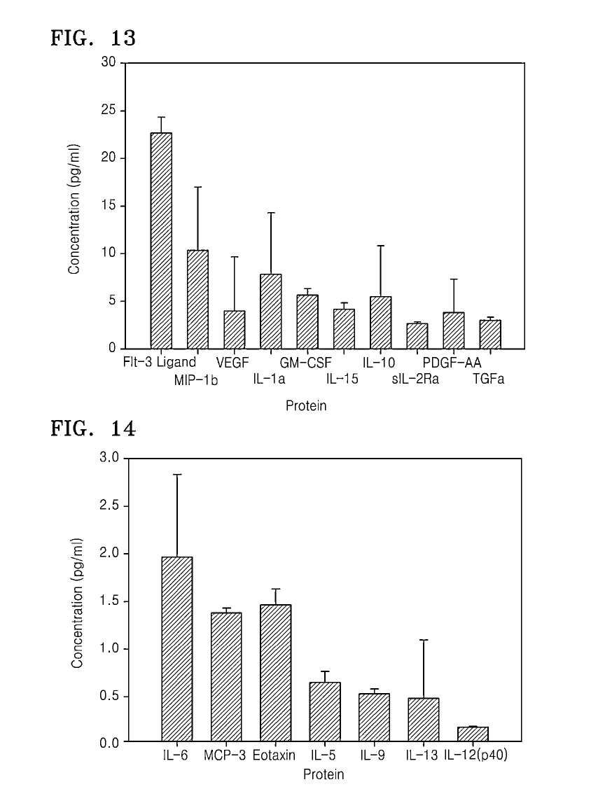

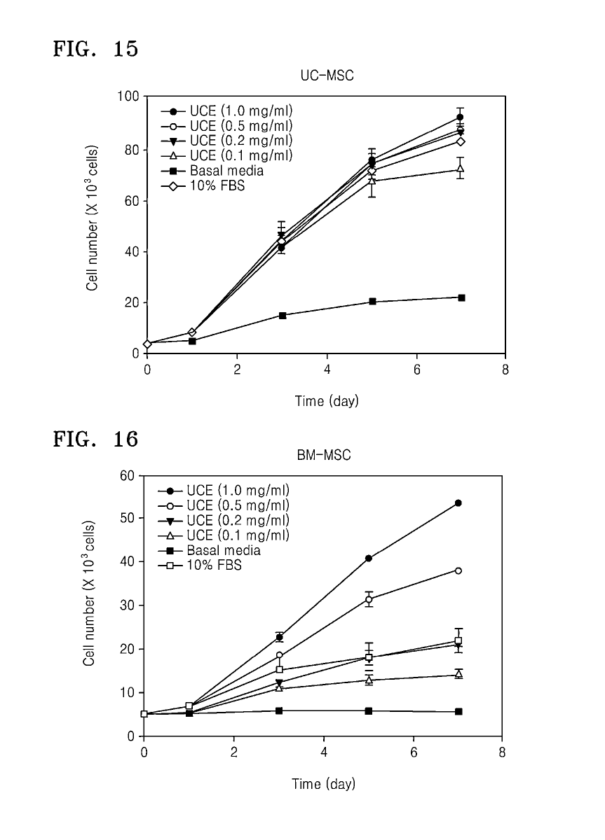

When the cut umbilical cord was stirred in PBS having a weight that is twice as great as the umbilical cord, a total of 53.76 mg (3.16 mg/ml) of protein, in PBS having a weight that is three times as great, a total of 64.23 mg (2.47 mg/ml) of protein, in PBS having a weight that is five times as great, and a total of 73.78 mg (1.48 mg/ml) of protein were eluted and thus, it may be concluded that as the amount of PBS increases, the total amount of protein eluted increases as well (FIG. 11). As the total amount of PBS increases, the total amount of protein increases as well, but a protein concentration of the final UCE decreases. Thus, a separate concentration process is needed to produce protein at an optimal concentration and the eluted protein may be lost during this process.

### EXAMPLE 7

### Qualitative and Quantitative Analyses of Primary Proteins of UCE

To analyze the types and comparative quantity of primary ingredients such as growth factors, chemokines, and cytokines among the proteins included in an UCE, a RayBio Human cytokine array kit capable of analyzing 507 different types of proteins was used. Three donors donated 1 mg of three different types of UCEs, which were membrane treated and then reacted. A dot detected therefrom was analyzed for dot intensity by using a MultiGauge program.

The results therefrom are shown in the drawings and tables.

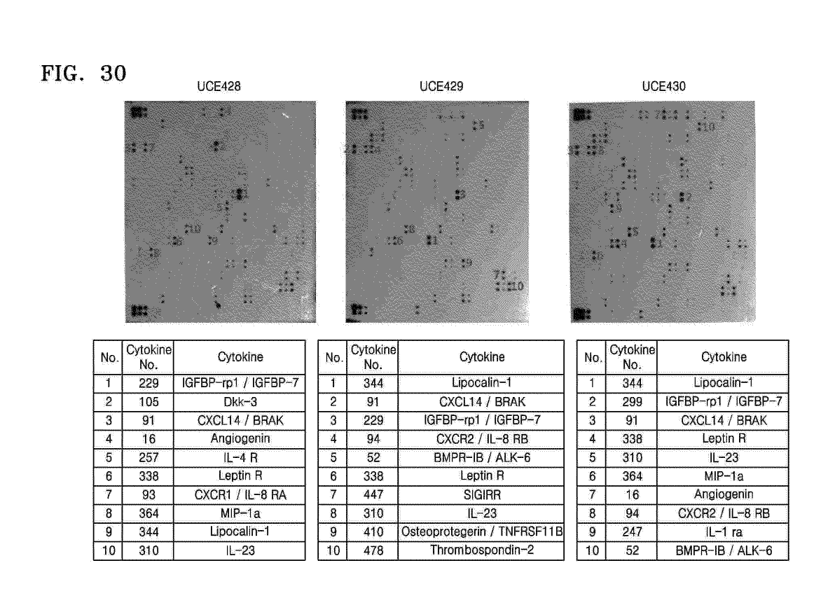

FIG. 30 shows cytokine array results for 507 different types of human cytokines, chemokines, and growth factors included in extracts separated from the umbilical cords of three different donors and 10 cytokines that are included in greatest amounts in the extracts from different donors.

FIG. 31 is a comparative quantitative graph showing a material that is commonly included in an extract separated from three different umbilical cords. The materials that are commonly included in greatest amounts are IGFBP-7, lipocalin-1, or the like.

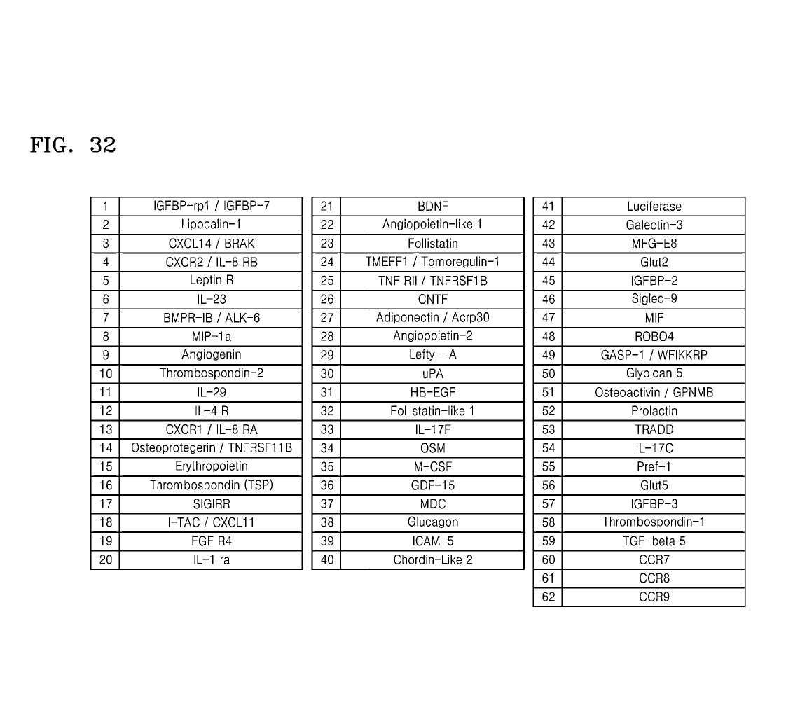

FIG. 32 sequentially shows 62 types of human cytokines identified in three different types of umbilical cords that are included in the greatest amount to the smallest amount.

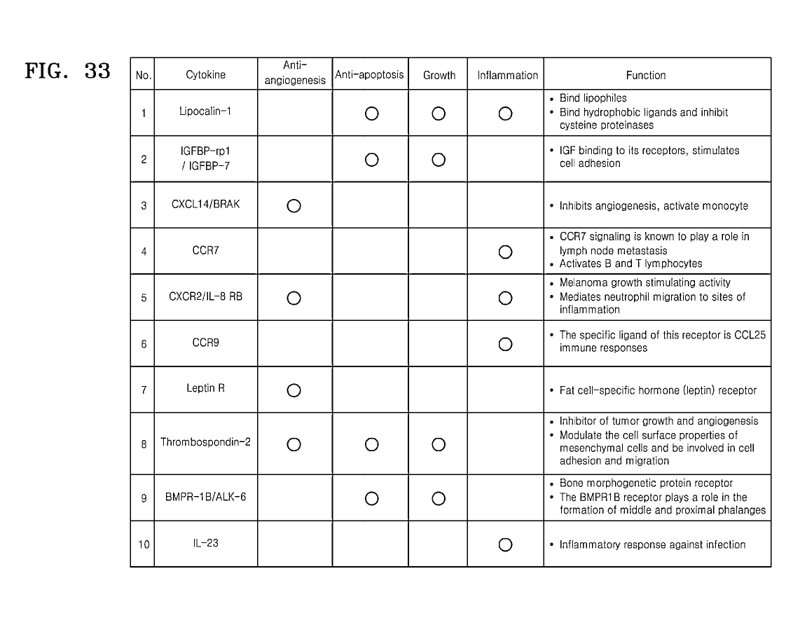

FIG. 33 describes functions of 10 cytokines having functions that are well known in a human body.

A quantitative analysis was performed on 42 types of well-known cytokines in the UCE. 1 mg/ml of a UCE sample was subjected to antibody-antigen reactions with 42 types of cytokines by using a MILLIPLEX™ Human Cytokine/Chemokine panel (42-plex: EGF, Eotaxin, FGF-2, Flt-3 Ligand, Fractalkine, G-CSF, GM-CSF, GRO, IFNα2, IFNγ, IL-1ra, IL-1α, IL-1β, IL-2, sIL-2Rα, IL-3, IL-4, IL-5, IL-6, IL-7, IL-8, IL-9, IL-10, IL-12(p40), IL-12(p70), IL-13, IL-15, IL-17, IP-10, MCP-1, MCP-3, MDC, MIP-1α,MIP-1β, PDGF-AA, PDGF-AB/BB, RANTES, sCD40L, TGFα, TNFα, TNFβ, and VEGF; a product of Millipore) and then subjected to a quantitative analysis by using a Luminex 200 System.

As a result of the cytokine quantitative analysis, it was identified that an average of 1,400 pg/ml of FGF-2, 1,480 pg/ml of G-CSF, 860 pg/ml of MCP-1, 900 pg/ml of GRO, 700 of pg/ml IL-1ra, and 620 pg/ml of IP-10 are primary ingredients among extracted cytokines (FIG. 12).

### EXAMPLE 8

### Cell Proliferation According to Culturing Additives of a Basal Medium

UCE, in which serum was removed, was treated at a concentration of 0, 0.1, 0.2, 0.5, or 1 mg/ml. As a control group, in a medium including 10% serum (SH30919.03, a product of Hyclone) UC-MSCs, bone marrow-derived stem cells (BM-MSC), and skin fibroblasts were cultured and then 10% WST-1 assay (EZ-3000, a product of Daeillab) was performed for every two days for a total of 7 days. A WST-1 assay reagent was added such that the reagent is 10% of the medium, reacted for about 1 hour under the same conditions as culturing conditions, and then absorbance was measured at 450 nm.

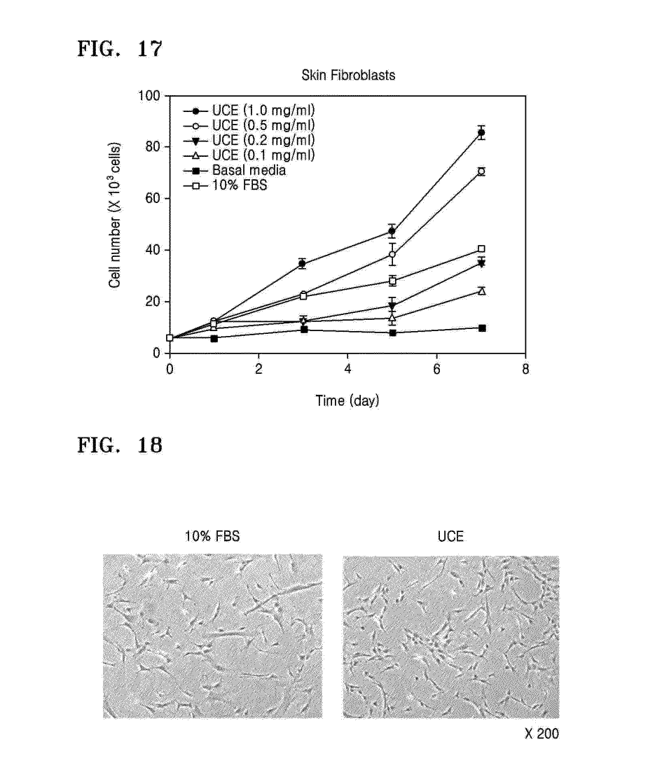

From the results of WST-1 assay, when the UCE was treated in a basal medium at different concentrations, it was identified that UC-MSCs, BM-MSCs, and skin fibroblasts at a concentration of 0.2 mg/ml showed cell proliferation at the same rate as cells in a medium including 10% FBS, and when the UCE was treated at a concentration of 0.5 mg/ml or greater, it was identified that cells proliferated more than the cells in the medium including 10% FBS (FIG. 15-17).

Also, cells grown in a basal medium including the UCE were found to have smaller morphology during the cell culturing than the cells grown in the culture medium including 10% FBS (FIG. 18).

### EXAMPLE 9

### Maintenance of Differentiation Potency of Stem Cells in an UCE

In a medium including 10% FBS and 0.2 mg/ml of UCE, bone marrow and UC-MSCs were cultured, and the product obtained therefrom was treated with mesenchymal stem cell markers, and then subjected to fluorescence activated cell sorter (FACS) analysis.

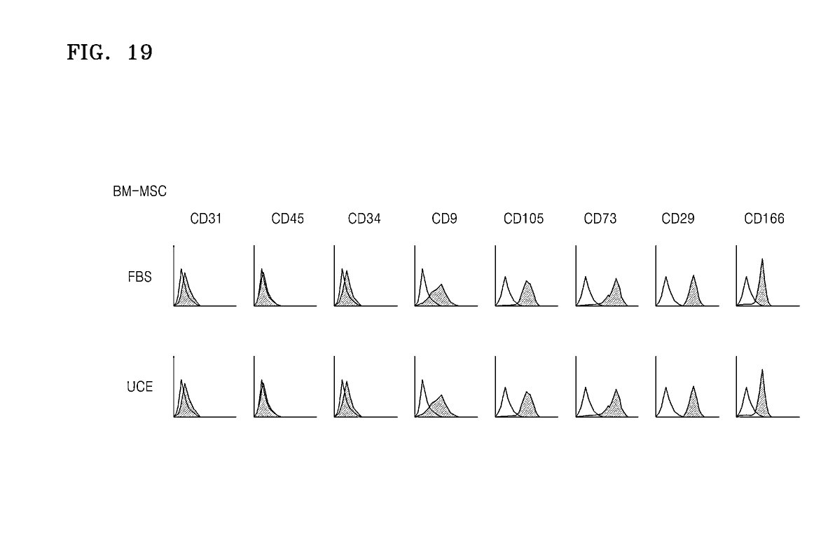

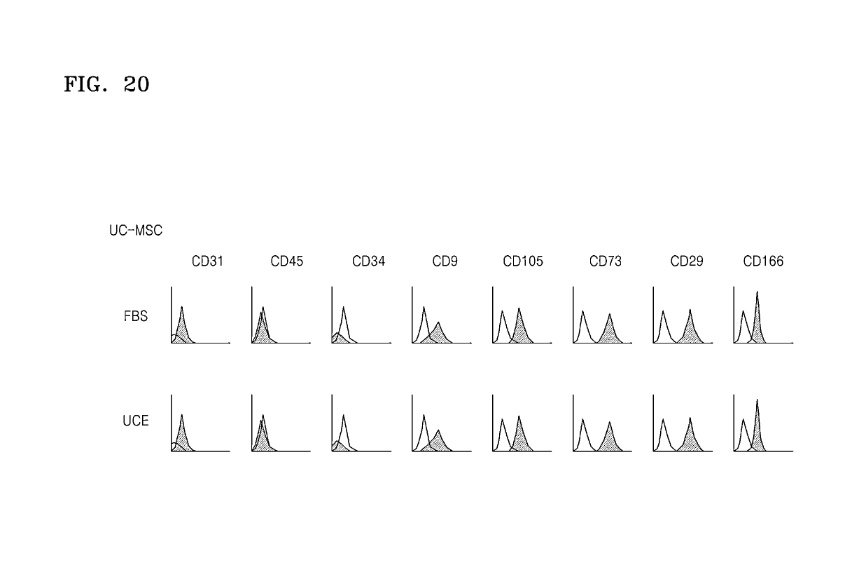

As a result of the FACS analysis, the stem cells cultured in a basal medium including the UCE were found to maintain mesenchymal stem cell specific cell surface markers and thus, the stem cells were found not to show changes to stem cell properties such as differentiation (FIGS. 19 and 20).

Also, the UCE was used to continuously subculture the stem cells at intervals of three days of doubling time to compare stemness of the stem cells to the stem cells cultured in the medium including 10% FBS.

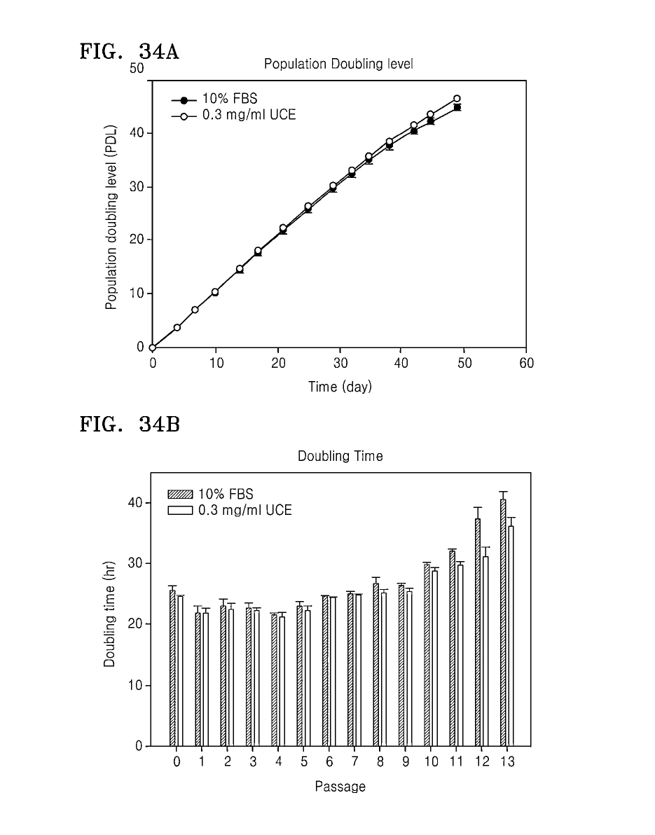

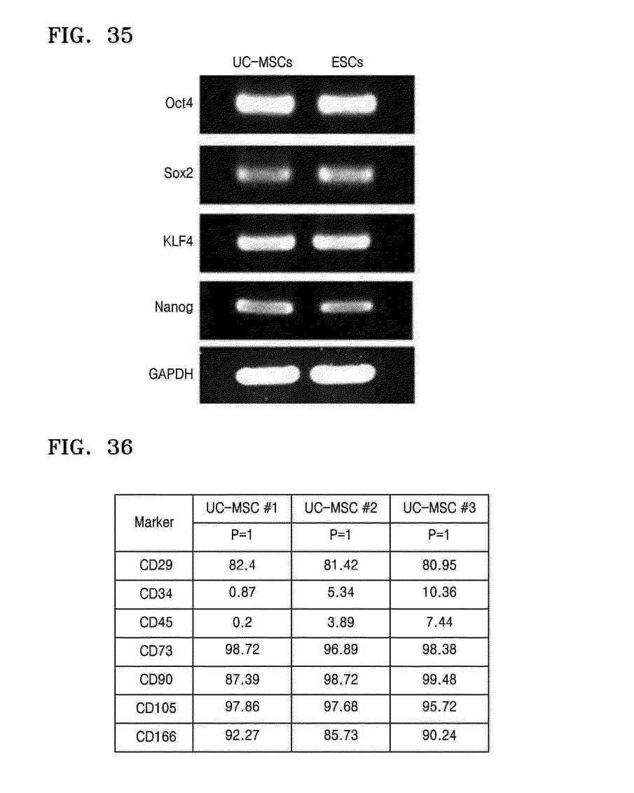

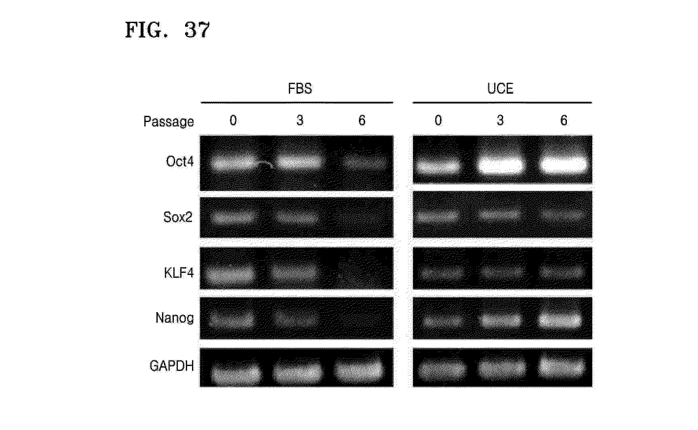

The results thereof are as shown in FIGS. 34 and 35. FIGS. 34 and 35 show results of experiments comparing stemness maintenance between stem cells that are continuously subcultured in UCE and in a medium including 10% FBS, wherein doubling time (Td) values of the stem cells during an initial subculture and after 10 cycles of subcultures are compared.

The proliferation rate and Td of the stem cells grown in a medium including 0.3 mg/ml UCE and without 10% FBS were not much different from the proliferation rate and Td of the stem cells grown in a medium including 10% FBS

and actually proliferated at a higher rate by a small difference. The concentration of 0.3 mg/ml of UCE was selected such that the stem cells may show a similar level of propagation as the 10% FBS. The stem cells were proliferated at a faster rate than the stem cells that were treated at a higher concentration of the UCE.

### EXAMPLE 10

### Preparation of UCE and Collagen

An umbilical cord was cut into a length of about 1 cm to about 2 cm, and then washed with Dulbecco's PBS (DPBS) twice or more. Then, the umbilical cord was treated with a 70% ethanol solution, reacted at a temperature of 4° C. for 1 hour, and then washed with distilled water twice or more to weigh the umbilical cord. Then, the umbilical cord was treated with DPBS having a weight that is about three times as great as the weight of the umbilical cord, treated at a temperature of 4° C. for 24 hours, and then UCE was collected therefrom. The collected UCE was filtered by using a final filter having a diameter of 0.22 μm and then stored at a temperature of 4° C.

A 3% H₂O₂ solution was added to a residual umbilical cord and then treated at a temperature of 4° C. for about 12 hours to about 24 hours. Then, the residual umbilical cord was washed with distilled water twice or more until foams disappeared. Then, the residual umbilical cord was treated with a 0.5 M acetic acid solution at a weight that is about 10 times as great as the weight of the umbilical cord and tissues of the residual umbilical cord were pulverized by using a blender and a homogenizer. The residual umbilical cord was treated with 10% of pepsin based on weight and then reacted at a temperature of 4° C. for 24 hours. The residual umbilical cord was centrifuged at 10,000 rpm and at a temperature of 4° C. for 30 minutes. After the centrifugation, NaOH was used to set a pH of the supernatant obtained therefrom at 7 to eliminate the activity of the pepsin enzyme. A volume of the pH-adjusted solution was measured and then treated with NaCl to make 2.4 M, based on the volume of the pH-adjusted solution. The pH-adjusted solution was stirred until all NaCl was dissolved and then left to stand for about 12 hours to about 24 hours at a temperature of 4° C. until collagen salted out and precipitated. After centrifuging the pH-adjusted solution at 10,000 rpm and at a temperature of 4° C. for 30 minutes, salted out collagen pellets were separated and then weighed. The collagen pellets were diluted in distilled water at a weight that is 10 times as great as the weight of the collagen pellets, and the diluted collagen pellets were desalinated and concentrated by using an ultrafiltration system. Finally, the desalinated collagen pellets were removed of microorganisms by filtration, freeze-dried, and then stored.

The UCE collected therefrom was quantified by Bradford analysis, and the collagen prepared as described above was quantified by hydroxyproline analysis.

### EXAMPLE 11

### Culturing Cells with UCE and Collagen Coating in a Basal Medium

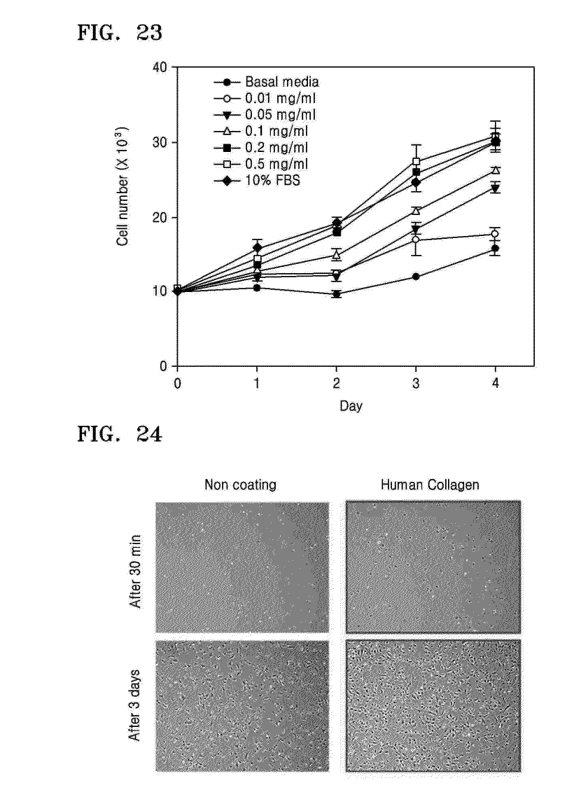

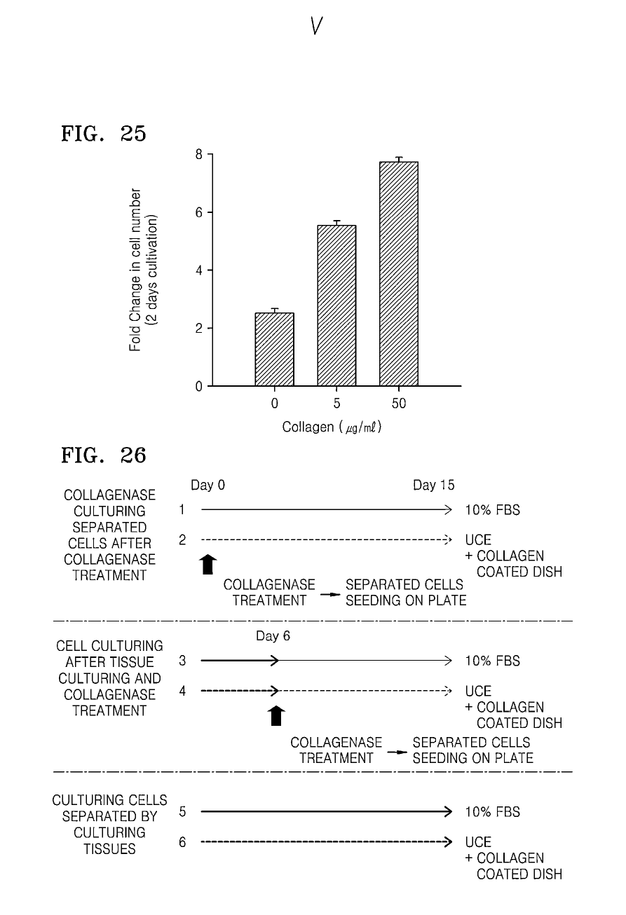

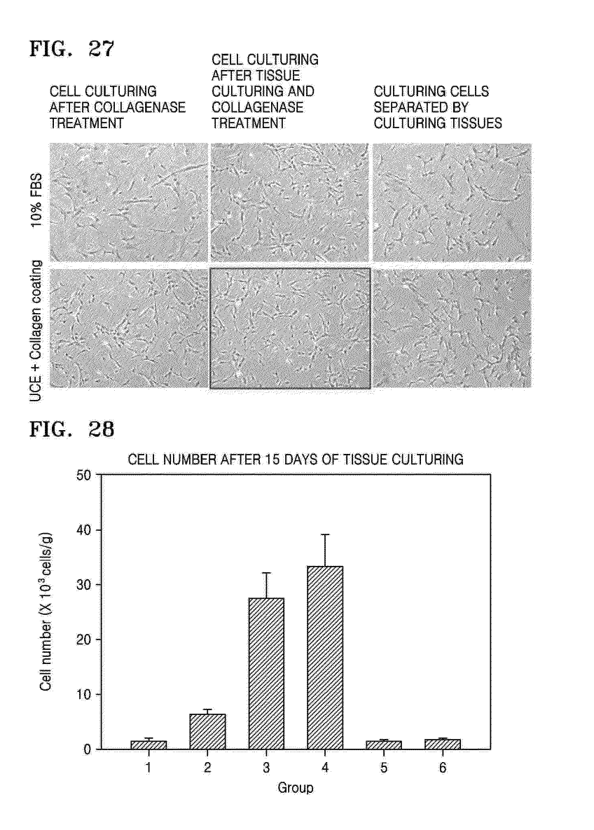

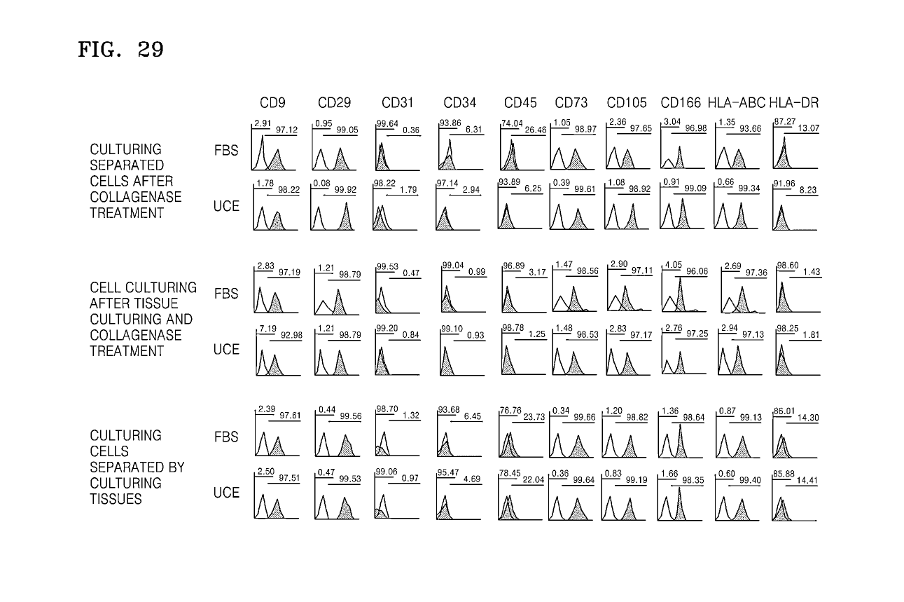

Collagen was dissolved in D.W. at a concentration of 50 μg/ml and then treated on a culture dish to coat the same in an incubator for 1 hour. After the coating, a collagen solution was removed therefrom, washed with a phosphate buffer twice or more, completely dried at room temperature, and then cells were cultured. The UCE was treated at a concentration of 0, 0.1, and 0.2 mg/ml in a basal medium to culture cells, a medium including 10% FBS was used as a control group, and WST-1 assay (EZ-3000, a product of Daeillab) was performed for every two days for a total of 7 days to compare cell growth. A WST-1 assay reagent was added to a medium at 10% and then reacted under the same culture conditions for about 1 hour, and absorbance was measured at 450 nm (FIGS. 23 and 24).

### EXAMPLE 12

### Comparison of Cell Recovery Rate and Proliferation of Umbilical Cord Stem Cells According to Methods of Separation

Blood external to an umbilical cord was removed by DPBS without Ca²⁺ and Mg²⁺, an external amnion was removed and two arteries were removed from the umbilical cord to compare six cell separation methods described below.

<12-1>The First Separation Method of Umbilical Cord Stem Cells

Tissues were treated with collagenase and cells were cultured in a medium including 10% FBS. In greater detail, tissues removed of blood were cut into a size of 1 mm³, treated with α-MEM, including 200 U/ml of collagenase type I, for 5 hours to separate cells, and 2×10³ of the cells were disposed per 1 cm² of the culturing dish including α-MEM in which 100 U/ml of penicillin, 0.1 μg/ml of streptomycin, and 10% FBS were included, to culture the cells in an incubator in which 5% of CO₂ was supplied at a temperature 37° C.

<12-2>The Second Separation Method of Umbilical Cord Stem Cells

After treating tissues with collagenase, cells were cultured in a medium including 0.2 mg/ml of UCE. In greater detail, tissues removed of blood were cut into a size of 1 mm³, treated with α-MEM, including 200 U/ml of collagenase type I, for 5 hours to separate cells, and 2×10³ of the cells were disposed per 1 cm² of the culturing dish including α-MEM in which 100 U/ml of penicillin, 0.1 μg/ml of streptomycin, and 10% FBS were included, to culture the cells in an incubator in which 5% of CO₂ was supplied at a temperature 37° C.

<12-3>The Third Separation Method of Umbilical Cord Stem Cells

Tissues were cultured in a medium including 10% FBS and cells were cultured in a medium including 10% FBS. In greater detail, tissues removed of blood were cut into a size of 1 mm³ and then put into α-MEM including 100 U/ml of penicillin, 0.1 μg/ml of streptomycin, and 10% of FBS, the tissues were cultured for 7 days, and when cells appeared to adhere to the bottom, the cells were treated with 200 U/ml of α-MEM including collagenase type I for 4 hours until all of the extracellular matrix was dissolved and then the product obtained therefrom was centrifuged and washed with PBS to only separate the cells. Thereafter, 2×10³ of the cells were disposed per 1 cm² of the culturing dish including α-MEM in which 100 U/ml of penicillin, 0.1 μg/ml of streptomycin, and 10% FBS were included, to culture the cells in an incubator in which 5% of CO₂ was supplied at a temperature 37° C.

<12-4>The Fourth Separation Method of Umbilical cord Stem Cells

Tissues were cultured in a medium including 0.2 mg/ml of UCE, treated with a collagenase, and then cells were cultured in a medium including 0.2 mg/ml of UCE. In greater detail, tissues removed of blood were cut into a size of 1 mm³ and then put into a collagen-coated dish to put the cells in α-MEM including 100 U/ml of penicillin, 0.1 μg/ml of streptomycin, and 10% of FBS, the tissues were cultured for 7 days, and when cells appeared to adhere to the bottom, the cells were treated with 200 U/ml of α-MEM including collagenase type I for 4 hours. Thereafter, 2×10³ of the cells were disposed per 1 cm² of a collagen-coated culture dish including α-MEM in which 100 U/ml of penicillin, 0.1 μg/ml of streptomycin, and 0.2 mg/ml of UCE were included, to culture the cells in an incubator in which 5% of CO₂ was supplied at a temperature 37° C.

<12-5>The Fifth Separation Method of Umbilical Cord Stem Cells

Tissues were cultured in a medium including 10% FBS. In greater detail, tissues removed of blood were cut into a size of 1 mm³ and then put into α-MEM including 100 U/ml of penicillin, 0.1 μg/ml of streptomycin, and 10% of FBS, and then cultured in an incubator in which 5% of CO₂ was supplied at a temperature of 37° C.

<12-6>The Sixth Separation Method of Umbilical Cord Stem Cells

Tissues were cultured in a medium including 0.2 mg/ml of UCE. In greater detail, tissues removed of blood were cut into a size of 1 mm³ and then put into α-MEM including 100 U/ml of penicillin, 0.1 μg/ml of streptomycin, and 10% of FBS, and then cultured in an incubator in which 5% of CO₂ was supplied at a temperature of 37° C.

<Example 13>Identification of ES Cell Markers through RT-PCR

Cell pellets were washed with DPBS without Ca²⁺ and Mg²⁺, 1 ml of lysis buffer (a product of iNtRON Biotechnology) was added thereto and a total RNA was separated therefrom according to the method described in the manual available from iNtRON Biotechnology. 1 μg of RNA was reverse transcribed by using a cDNA synthesis kit (a product of iNtRON Biotechnology) in a 20 μL of a reaction solution including a reaction buffer, 1 mM of dNTP mixture, 0.5 μg/μL of oligo(dT)15, 20 U of RNase inhibitor, and 20 U of AMV reverse transcriptase. The reaction was performed at a temperature of 42° C. for 60 minutes. The RT products (cDNAs) obtained therefrom were subjected to PCR by using a 2× PCR Master mix solution kit (a product of iNtRON Biotechnology) including 10 μL of a reaction solution including 1× Taq buffer, 0.25 U of Taq polymerase, 10 pM of sense and antisense gene-specific primers. The amplification was performed for a total of 32 cycles and each cycle included 30 seconds of denaturation at a temperature of 94° C., 30 seconds of annealing, and 30 seconds of extension at a temperature of 72° C. After completing the reaction, the PCR products obtained therefrom were loaded in a 2% agarose gel for electrophoresis. After the electrophoresis, the gel was stained with ethidium bromide and an image of DNA was obtained by using ultraviolet rays.

TABLE 1
DNA sequence information of primers

| Genes | | Primer sequences (5′-3′) | | Temperature (° C.) |
|---|---|---|---|---|
| OCT 4 | Sense | agaaggagtggtccgagtg | SEQ ID NO: 1 | 60 |
| | Antisense | agagtggtgacggagacagg | SEQ ID NO: 2 | |
| Nanog | Sense | atacctcagcctccagcaga | SEQ ID NO: 3 | 59 |
| | Antisense | cctgattgrrccaggattgg | SEQ ID NO: 4 | |
| KLF4 | Sense | accctgggtcttgaggaagt | SEQ ID NO: 5 | 59 |
| | Antisense | tgccttgagatgggaactct | SEQ ID NO: 6 | |
| Sox2 | Sense | gatgcacaactcggagatcag | SEQ ID NO: 7 | 60 |
| | Antisense | gccgttcatgtaggtctgcga | SEQ ID NO: 8 | |
| GAPDH | Sense | gaaggtgaaggtcggagtca | SEQ ID NO: 9 | 60 |
| | Antisense | ggaggcattgctgatgatct | SEQ ID NO: 10 | |

### EXAMPLE 14

### Expression Analysis of Mesenchymal Stem Cell Markers through FACS Analysis

Flow cytometry was used to analyze properties of separated cells. The separated cells were washed by using PBS for the flow cytometry, treated with trypsin-EDTA to make a monoclonal cell group, and then washed with PBS including 2% FBS. Thereafter, matrix receptors (CD44 and CD105), respectively bound to fluorescein isothiocyanate (FITC) or phycoerythrin (PE); integrin (CD29); PECAM (CD31); VCAM-1 (CD106); SH2 (CD105); SH3 and SH4 (CD73); Thy-1 (CD90); hematopoietic markers (CD14 and CD34); and MHC markers (HLA-DR and HLA-Class I) were reacted for 20 minutes and then analyzed through a flow cytometry system (FACSCalibur, a product of Becton-Dickinson).

### EXAMPLE 15

### Evaluation of a Filler Including UCE

UCE was mixed at a concentration of 500 ug/ml in a hyaluronic acid derivative to prepare a mixture and then a rodent (BALB/c-nuSIc, female, and 5 weeks old) was treated with the mixture. Each treatment group was subcutaneously injected with 200 ul of the mixture and samples were extracted after 1 week, 4 weeks, 8 weeks, and 12 weeks. The rodent was subjected to a cervical vertebra dislocation and all tissues adhered around the samples were removed, the weight of each sample was weighed, and then fixed with 4% neutral buffered formalin to perform hematoxylin & eosin staining.

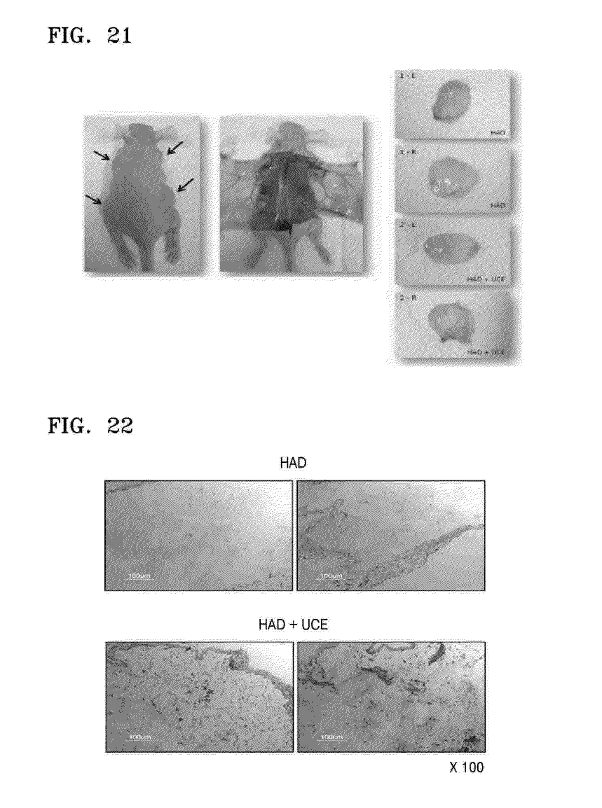

As a result, it was found that when the UCE is mixed into a filler for tissue restoration, the UCE attracts nearby tissues to maintain wound healing effects (FIGS. 21 and 22).

The UCE according to an embodiment may be used as a serum substitute for culturing animal-derived cells and stem cells. Also, the UCE according to the present invention may be used in a filler or a dressing for tissue restoration, and a cosmetic composition for improving skin conditions.

As described above, according to the one or more of the above embodiments of the method of separating and culturing UC-MSCs, stem cells having excellent proliferation and differentiation potency may be maximally obtained in a short period of time (15 days) by using a medium without FBS (about 2.0×10⁸ cells), and a great number of cells may be separated from only two or three times of sub-culturing (about 1.0×10¹° cells from 50 g of umbilical cord tissues) and thus, the method may be useful for the development of future stem cell therapy products.

It should be understood that the exemplary embodiments described therein should be considered in a descriptive sense only and not for purposes of limitation. Descriptions of features or aspects within each embodiment should typically be considered as available for other similar features or aspects in other embodiments.

While one or more embodiments of the present invention have been described with reference to the figures, it will be understood by those of ordinary skill in the art that various changes in form and details may be made therein without departing from the spirit and scope of the present invention as defined by the following claims.

1. A method of preparing a mammalian umbilical cord extract, the method comprising:
cutting an umbilical cord;
putting the umbilical cord into a buffer;
stirring the umbilical cord impregnated in the buffer;
and centrifuging a product obtained from the stirring to obtain a supernatant.

2. The umbilical cord extract prepared according to claim 1.

3. The umbilical cord extract of claim 2, wherein the umbilical cord extract comprises proteins of insulin-like growth factor binding protein-7 (IGFBP-7), Lipocallin-1, CXCL14, Leptin R, IL-23, MIP-1a, Angiogenin, Thrombospondin-2, IL-29, and IL-4R.

4. A serum substitute comprising an umbilical cord extract.

5. The serum substitute of claim 4, wherein the umbilical cord extract is prepared according to a method, comprising:
cutting an umbilical cord;
putting the umbilical cord into a buffer;
stirring the umbilical cord impregnated in the buffer; and
centrifuging a product obtained from the stirring to obtain a supernatant.

6. A cell culture medium comprising the serum substitute of claim 4.

7. The cell culture medium of claim 6, wherein the cell is an animal cell.

8. The cell culture medium of claim 7, wherein the animal cell is a stem cell.

9. The cell culture medium of claim 8, wherein the stem cell is an umbilical cord-derived stem cell.

10. A filler for tissue restoration comprising an umbilical cord extract.

11. The filler for tissue restoration of claim 10, wherein the umbilical cord extract is prepared according to a method, comprising:
cutting an umbilical cord;
putting the umbilical cord into a buffer;
stirring the umbilical cord impregnated in the buffer; and
centrifuging a product obtained from the stirring to obtain a supernatant.

12. A method of separating stem cells from a mammalian umbilical cord, the method comprising:
putting an umbilical cord tissue morcellated with a cell culture medium composition including a mammalian umbilical cord extract into a cell culture container;
treating the umbilical cord tissue with an enzyme; and
separating stem cells from the umbilical cord tissue.

13. The method of claim 12, wherein the cell culture container is coated with a cell adhesion protein.

14. The method of claim 12, wherein the cell adhesion protein is selected from the group consisting of a mammalian placenta-derived collagen, gelatin, fibronectin, laminin, and poly-D-lysin.

15. A method of culturing stem cells, the method comprising:
adding an umbilical cord extract to a stem cell culture medium; and
culturing stem cells by using the stem cell culture medium in a cell culture container.

16. The method of claim 15, wherein the cell culture container is coated with a cell adhesion protein.

17. The method of claim 15, wherein the stem cell is a tissue-derived stem cell.

18. The method of claim 16, wherein the tissue is selected from the group consisting of fat, an umbilical cord, a liver, and periosteum.

19. The method of claim 15, wherein the stem cell culture medium does not include serum.

20. The method of claim 15, wherein the cell adhesion protein is selected from the group consisting of a mammalian placenta-derived collagen, gelatin, fibronectin, laminin, and poly-D-lysin.

21. The method of claim 15, wherein the stem cell is a cell in which at least one gene from the group consisting of Oct 4, Sox2, KLF4, and Nanog is expressed.

22. The method of claim 15, wherein the stem cell is selectively positive for CD29, CD73, CD90, CD105, and CD166, and is selectively negative for CD34 and CD45.

23. The method of claim 15, wherein when the stem cell is cultured by using an umbilical cord extract, the stem cell continuously expresses at least one gene from the group consisting of Oct4, Sox2, KLF4, and Nanog, which are embryonic stem cell specific genes, even when the cells are subcultured.


SEQUENCE LISTING (10 sequences)

SEQ ID NO: 1 - DNA, 19 bp, Artificial Sequence
  forward primer for OCT 4
agaaggagtggtccgagtg

SEQ ID NO: 2 - DNA, 20 bp, Artificial Sequence
  reverse primer for OCT 4
agagtggtgacggagacagg

SEQ ID NO: 3 - DNA, 20 bp, Artificial Sequence
  forward primer for Nanog
atacctcagcctccagcaga

SEQ ID NO: 4 - DNA, 20 bp, Artificial Sequence
  reverse primer for Nanog
cctgattgrrccaggattgg

SEQ ID NO: 5 - DNA, 20 bp, Artificial Sequence
  forward primer for KLF4
accctgggtcttgaggaagt

SEQ ID NO: 6 - DNA, 20 bp, Artificial Sequence
  reverse primer for KLF4
tgccttgagatgggaactct

SEQ ID NO: 7 - DNA, 21 bp, Artificial Sequence
  forward primer for Sox2
gatgcacaactcggagatcag

SEQ ID NO: 8 - DNA, 21 bp, Artificial Sequence
  reverse primer for Sox2
gccgttcatgtaggtctgcga

SEQ ID NO: 9 - DNA, 20 bp, Artificial Sequence
  forward primer for GAPDH
gaaggtgaaggtcggagtca

SEQ ID NO: 10 - DNA, 20 bp, Artificial Sequence
  reverse primer for GAPDH
ggaggcattgctgatgatct